(19) United States
(12) Patent Application Publication
(10) Pub. No.: US 2024/0000293 A1
(43) Pub. Date: Jan. 4, 2024

(54) MEDICAL IMAGING METHOD AND DEVICE

(71) Applicant: ARIEL SCIENTIFIC INNOVATIONS LTD., Ariel (IL)

(72) Inventor: David ABOOKASIS, Kfar-Saba (IL)

(21) Appl. No.: 18/036,004

(22) PCT Filed: Nov. 9, 2021
(86) PCT No.: PCT/IL2021/051328

§ 371 Date: May 9, 2023

Related U.S. Application Data
(60) Provisional application No. 63/111,120, filed on Nov. 9, 2020.

(51) Int. Cl. A61B 1/00; A61B 1/04

(52) U.S. Cl. CPC A61B 1/0005; A61B 1/042; A61B 1/00186

(57) ABSTRACT

Disclosed are methods and devices suitable for providing diagnostic images. In some embodiments, an imaging device suitable for use with a medical imaging device such as an endoscope is disclosed. In some embodiments, a diagnostic image and a method of making such a diagnostic image is provided.

### CROSS-REFERENCE TO RELATED APPLICATIONS

This application claims the benefit of priority of U.S. Provisional Application Ser. No. 63/111,120, filed Nov. 9, 2020, the content of which is incorporated by reference herein by reference in its entirety.

### FIELD AND BACKGROUND OF THE INVENTION

The invention, in some embodiments, relates to the field of medical imaging, and more particularly but not exclusively, to methods and devices suitable for providing diagnostic medical images.

In the field of medicine it is known to provide a diagnostic image of tissue.

In a first step, tissue is illuminated with light from a light source and a pixelated camera is used to concurrently acquire an image of the illuminated tissue.

Illumination is with any suitable light source, e.g., a white light source, a narrow band light source, a monochromatic light source that illuminates the tissue with a single discrete wavelength of light or polychromatic light source that illuminates the tissue with two or more discrete wavelengths of light.

Image acquisition is with any suitable pixelated camera, e.g., a multispectral camera, a color camera (e.g., RGB) or a monochrome camera.

In a second step, a diagnostic image is provided.

In some instances, the provided diagnostic image is the acquired image with little or no post-acquisition processing. For example, acquisition of an image of tissue while illuminating the tissue with specific wavelengths of light can reveal diagnostically-useful features of the tissue.

In some instances, the provided diagnostic image is a false-color diagnostic image that is generated from one or more acquired images, the false color revealing diagnostically-useful features of the tissue, see for example, US patent publication 2014/0180129.

A provided diagnostic image is subsequently used in any number of useful ways.

In some instances, a provided diagnostic image is displayed, optionally in real time, allowing a person, typically a medical professional, to look at the image to receive useful information therefrom. Typically, the useful information helps the person make a decision. For instance, in some instances the displayed diagnostic image constitutes evidence indicating the possible presence of a pathology. In some instances, a provided diagnostic image is displayed as a spatial-domain image. In some instances, a diagnostic image is processed prior to display, e.g., a histogram of the provided diagnostic image is calculated and displayed or the provided diagnostic image undergoes a Fourier Transform and the Fourier domain image is displayed.

Diagnostic images (with or without processing) are displayed in any suitable way including printing on a tangible medium (paper, film) or displayed on a display screen (e.g., LCD, LED, projector screen).

In some instances, a provided diagnostic image is automatically analyzed (e.g., by a computer or other electronic device) to identify noteworthy features that are medically significant, e.g., the possible presence of a pathology. In some instances, automatic analysis is performed by identifying pixel values, in some instances values of a group of pixels, that are potentially medically significant. In some instances, automatic analysis is performed by comparing two images of the same tissue, e.g., taken at different dates or under two different conditions).

In some instances, a diagnostic image is stored (e.g., electronically on a storage medium such as a hard disk or flash memory) for future use, locally or remotely (e.g., cloud storage).

It would be useful to have methods and devices that are useful for providing diagnostic medical images.

### SUMMARY OF THE INVENTION

Some embodiments of the invention relate to methods and devices suitable for providing diagnostic medical images. In some embodiments, an imaging device suitable for use with a medical imaging device such as an endoscope is provided. In some embodiments, a diagnostic image and a method of making such a diagnostic image is provided.

According to an aspect of some embodiments of the teachings herein, there is provided a method for diagnosing a biological tissue comprising:
a) illuminating the tissue with multispectral band light;
b) receiving, at a first camera, a first multispectral band reflection signal from the biological tissue;
c) generating a multicoloured image from the first signal;
d) selecting a first property of the biological tissue;
e) selecting one or more first wavelengths based on the first property;
f) filtering a second multispectral reflection light signal to receive at the first camera a second signal comprising light at the one or more first selected wavelengths;
g) generating a first image from the filtered second multispectral light signal;
h) merging the first image with the multicolored image to create a first merged image; and
i) presenting the first merged image on a display.

In some embodiments, wherein filtering the one or more first wavelengths is by an optical filter, placed in front of the camera. In some embodiments, filtering the one or more first wavelengths is by selecting the one or more wavelengths in the second white light signal. In some embodiments, the first property is oxygen level, and the first one or more wavelengths include at least two wavelengths selected from green light at 520-560 nm. In some embodiments, the first property is deoxyhemoglobin level, and the first one or more wavelengths is at least one wavelength selected from red light at 600-700 nm. In some embodiments, the first property is perfusion of biological surfaces level, and the first one or more wavelengths is a single wavelength selected form 630-850 nm.

In some embodiments, the second signal is received by illuminating said biological tissue with a laser at the selected wavelength and generating the first image is generating a monochromatic image.

In some embodiments, the method may further include:
a) selecting a second property of the biological tissue;
b) selecting one or more second wavelengths based on said second property;
c) filtering a third multispectral reflection light signal to receive at the first camera a second signal comprising light at said second one or more selected wavelengths;
d) generating a second image from said third multispectral reflection light signal; and
e) merging said second image with the multicolored image to create a second merged image; and
f) presenting said second merged image on a display.

In some embodiments, the method may further include:
a) selecting a second property of the biological tissue;
b) selecting one or more second wavelengths based on said second property;
c) filtering said second multispectral reflection light signal to receive at said first camera also a second signal comprising light at the second one or more selected wavelengths; and
d) generating a second image from said second multispectral light signal;
e) merging said second monochromatic image with said first combined image to create a third combined image; and
f) presenting said third combined image on a display.

In some embodiments, the method may further include:
a) illuminating said biological tissue with a monochromatic light;
b) receiving, at a second camera, a monochromatic signal from said biological tissue;
c) generating a monochromatic image from said monochromatic signal;
d) merging said multicolored image with said monochromatic image to form a fourth merged image; and
e) displaying said fourth image on a display.

In some embodiments, the multispectral reflection light is white light. In some embodiments, illuminating said tissue with monochromatic light is by a laser source.

In some embodiments, the method may further include:
receiving, at a second camera, a white light reflection signal from the biological tissue; and
generating and presenting a white light image.

Some additional aspects of the invention may be directed to a system for diagnosing a biological tissue, comprising:
at least one multispectral light source;
at least one multispectral camera;
a filter configured to filter one or more wavelengths from a multispectral reflection light signal; and
and a controller configured to:
   a) control said multispectral light source to illuminate said tissue with multispectral band light;
   b) receive, from a first camera, a first multispectral band reflection signal from said biological tissue;
   c) generate a multicoloured image from said first signal;
   d) select a first property of said biological tissue;
   e) select one or more first wavelengths based on said first property;
   f) control said filter to filter a second multispectral reflection light signal to receive at said first camera a second signal comprising light at said one or more first wavelengths;
   g) generate a first image from said filtered second multispectral light signal;
   h) merge said first image with said multicolored image to create a first merged image; and
   i) present said first merged image on a display.

According to an aspect of some embodiments of the teachings herein, there is provided a method for generating an image of the surface of biological tissue, comprising:
a) receiving a first pixelated monochromatic image of an area of interest of the surface acquired during illumination of the surface with a first wavelength of light λ1;
b) receiving a second pixelated monochromatic image of the area of interest of the surface acquired during illumination of the surface with a second wavelength of light λ2, λ2 being different from λ1; and
c) generating a monochromatic third pixelated image from the first image and the second image by:
   for each desired location i of the area of interest, identifying a corresponding pixel P1(i) in the first image and a corresponding pixel P2(i) in the second image; and
   calculating a value for pixel P3(i) in the third image corresponding to the location i from a value of P1(i) and a value of P2(i), the value for P3(i) indicative of the relative amplitude scattering coefficient of tissue underlying the surface at the location i.

According to an aspect of some embodiments of the teachings herein, there is provided a method for generating an image of the surface of biological tissue, comprising:
a) acquiring a first pixelated monochromatic image of an area of interest of during illumination of the surface with a first wavelength of light λ1;
b) acquiring a second pixelated monochromatic image of the area of interest of during illumination of the surface with a second wavelength of light λ2, λ2 being different from λ1; and
c) receiving the first and second images and generating a monochromatic third pixelated image from the first image and the second image by:
   for each desired location i of the area of interest, identifying a corresponding pixel P1(i) in the first image and a corresponding pixel P2(i) in the second image; and
calculating a value for pixel P3(i) in the third image corresponding to the location i from a value of P1(i) and a value of P2(i), the value for P3(i) indicative of the relative amplitude scattering coefficient of tissue underlying the surface at the location i.

According to an aspect of some embodiments of the teachings herein, there is provided a device for generating an image of the surface of biological tissue, comprising a computer processor having at least one input port and at least one output port, the computer processor configured to:
a) receive a first pixelated monochromatic image of an area of interest of the surface acquired during illumination of the surface with a first wavelength of light λ1 through an input port;
b) receive a second pixelated monochromatic image of the area of interest of the surface acquired during illumination of the surface with a second wavelength of light λ2, λ2 being different from λ1 through an input port;
c) generate a monochromatic third pixelated image from the first image and the second image by:
   for each desired location i of the area of interest, identifying a corresponding pixel P1(i) in the first image and a corresponding pixel P2(i) in the second image; and
   calculating a value for pixel P3(i) in the third image corresponding to the location i from a value of P1(i) and a value of P2(i), the value for P3(i) indicative of the relative amplitude scattering coefficient of tissue underlying the surface at the location i.

In some embodiments, the device further comprises:
an illuminator for illuminating a surface with a first wavelength of light λ1 and with a second wavelength of light λ2;
a camera for acquiring a first image of an area of interest of a surface during illumination with the first wavelength of light λ1 by the illuminator and for providing an acquired first image to the computer processor through an input port; and
a camera for acquiring a second image of an area of interest of a surface during illumination with the second wavelength of light λ2 by the illuminator and for providing an acquired second image to the computer processor through an input port.

In some embodiments of the methods or the device, P3(i) is an approximation of the value of the slope of Rayleigh-Mie scattering coefficient as a function of wavelength.

In some embodiments of the methods or the device, P3(i) is related to the ratio
ΔP(i) to Δλ,
   where:
ΔP(i) is the difference between the values of P1(i) and P2(i); and
Δλ is the difference between λ1 and λ2.

In some embodiments of the method or the device, P3(i) is calculated using the formula:

(P1(i)−P2(i))/(λ1−λ2)

or a substantially-equivalent formula.

### BRIEF DESCRIPTION OF THE FIGURES

Some embodiments of the invention are herein described with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 (prior art) is a graph showing the dependence of Rayleigh and Mie scattering on wavelength in the visible spectrum and the size of the scattering particle, the graph adapted from Bin Omar AF and Bin MatJafri MZ in Sensors 2009, 9, 8311-8335;

FIGS. 2A and 2B schematically depict implementation of an embodiment of the method according to the teachings herein;

FIG. 3 is a schematic depiction of an embodiment of a device according to the teachings herein;

FIGS. 4A-4D illustrate aspects of an experiment performed by the Inventors to demonstrate the teachings herein:

FIG. 4A: schematic depiction of the experimental device used;

FIG. 4B: reproduction of an image of the surface of a mouse brain studied;

FIG. 4C: graphic depiction of the time-dependent variation in cerebral tissue oxygenation caused by oxygen deprivation;

FIG. 4D: graphic depiction of the time-dependent variation in cerebral tissue scattering according to the teachings caused by oxygen deprivation with accompanying images and corresponding histograms;

FIG. 5A is an illustration of a system for diagnosing a biological tissue according to some embodiments of the invention;

FIG. 5B is a flowchart of a method of diagnosing a biological tissue according to some embodiments of the invention; and

FIG. 6 is a block diagram of a computing device to be included in a system for diagnosing a biological tissue according to some embodiments of the invention.

### DESCRIPTION OF SOME EMBODIMENTS OF THE INVENTION

Some embodiments of the invention relate to methods and devices suitable for providing diagnostic medical images.

The principles and implementations of the teachings of the invention may be better understood with reference to the accompanying description and figures. Upon perusal of the description and figures present herein, one skilled in the art is able to implement the teachings of the invention without undue effort or experimentation. In the figures, like reference numerals refer to like parts throughout.

Before explaining at least one embodiment of the invention in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

The Inventors sought to invent novel methods and devices suitable for generating, and in some embodiments, acquiring and generating, diagnostic medical images. The Inventors now disclose that it is possible to generate a diagnostically-useful pixelated monochromatic image of an area of interest of tissue where the value of each pixel is indicative of the relative amplitude scattering coefficient of tissue underlying the surface. As known to a person having ordinary skill in the art, amplitude scattering coefficient is dependent on various properties of the materials that underly the surface such as the scattering cross-section of the materials, nuclear shape or size, protein density and others. In some embodiments, such an image can be used to help in identifying portions of tissue having anomalous scattering properties (when compared to surrounding tissue or to a reference image), such anomalous scattering properties being potentially indicative of some underlying pathology, such as the presence of a tumor. A person having ordinary skill in the art, e.g., a medical professional such as a radiologist, is able to use the fact that some portions of tissue have anomalous scattering properties as evidence to assist in diagnosing a pathology.

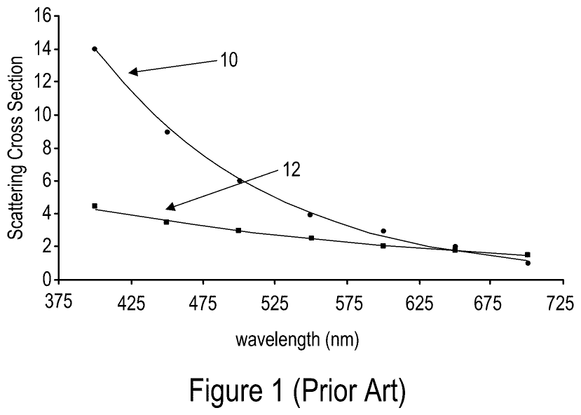

FIG. 1 is a graph showing the dependence of scattering cross section of 0.0285 micrometer-sized particles 10 (primarily Rayleigh scattering) and 0.2615 micrometer-sized particles 12 (primarily Mie scattering) on wavelength of visible light. It is seen that slopes of curves 10 and 12 are not constant, instead having a higher absolute value at lower wavelengths and a lower absolute value at higher wavelengths. The Inventors now disclose that by determining, for a location i, the intensity of scattering P1(i) at a wavelength λ1 and scattering P2(i) at a different wavelength λ2, an approximation of the value of the slope of the Rayleigh-Mie scattering coefficient as a function of wavelength for that location can be calculated. It can be concluded that the tissue underlying two locations having the same approximated slope have the same or similar relative amplitude scattering coefficients while two locations having a different approximated slope have substantially different relative amplitude scattering coefficients. When such a difference is identified in accordance with the teachings herein, locations having different material properties are identified, which different material properties may be clinically significant.

Thus, the Inventors now disclose that a diagnostic monochromatic pixelated image can be generated from two pixelated monochromatic images of an area of interest of a surface, a first image acquired at a first wavelength of light λ1 and a second image acquired at a second wavelength of light, λ2 being different from λ1.

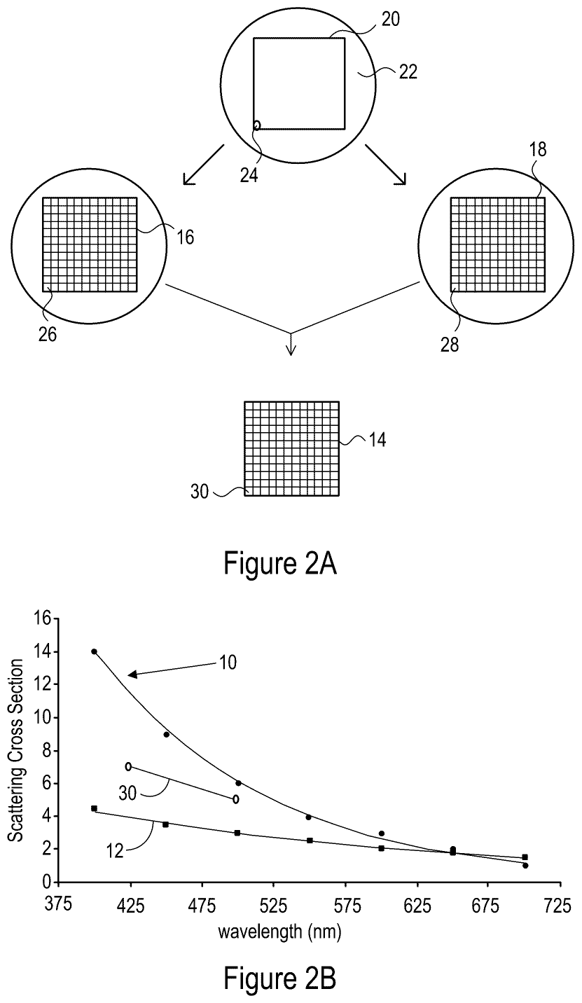

With reference to FIGS. 2A and 2B, the nature of a diagnostic image 14 according to the teachings herein and an embodiment of how such a diagnostic image can be generated from a first monochromatic pixelated image 16 and a second monochromatic pixelated image 18 is described for an area of interest 20 on a surface of biological tissue 22.

First image 16 of area of interest 20 is acquired at a first wavelength of light λ1 and second image 18 of area of interest 20 is acquired at a second wavelength of light λ2. First image 16 and second image 18 each comprises a 12×12 matrix of pixels, a total of 144 pixels each, each one of the 144 pixels corresponding to a different location i of area of interest 20. For each location i of area of interest 20, a corresponding pixel P1(i) in first image 16 and a corresponding pixel P2(i) in second image 18 are identified. In FIG. 2A a single location i labelled 24 and corresponding pixels P1(i) labelled 26 (in first image 16) and P2(i) labelled 28 (in second image 18) are indicated.

In accordance with an embodiment of the teachings herein, a value for a pixel P3(i) labelled 30 in the diagnostic image 14 corresponding to location i labelled 24 that is calculated from a value of pixel P1(i) labelled 26 in first image 16 and a value of pixel P2(i) labelled 28 in second image 18 is related to the ratio:
ΔP(i) to Δλ,
   where:
ΔP(i) is the difference between the values of P1(i) and P2(i) (i.e., [P1(i)−P2(i)] or [P2(i)−P1(i)]);
and
   Δλ is the difference between λ1 and λ2 (i.e., [λ1−λ2] or [λ2−1]),
   for example, P3(i) is calculated using the formula: (P1(i)−P2(i))/(λ1−λ2) or a substantially-equivalent formula. The term “substantially-equivalent formula” relates to a formula that includes a mathematical operation that does not qualitatively change the result, i.e., a mathematical operation designed to allow infringement of the claims. Such mathematical operations include, but are not limited to:
multiplication, division and/or exponentiation of any one of P1(i), P2(i), λ1, λ2, the numerator or the denominator by a number (constant or variable) small enough so as not to substantially change P3(i); and
addition or subtraction to any one of P1(i), P2(i), λ1, λ2, the numerator, or the denominator a number (constant or variable) small enough so as not to substantially change P3(i).

Without wishing to be held to any one theory, the Inventors believe that:
the value of P1(i) is related to the scattering cross section of particles in location i at λ1;
the value of P2(i) is related to the scattering cross section of particles in location i at λ2; and
the calculated value of P3(i) can be considered the slope of a linear approximation of the wavelength-dependence of Mie-Rayleigh scattering between λ1 and λ2. This is schematically depicted in FIG. 2B, with a line 30 drawn between a hypothetical P1(i) and P2(i) on a graph showing the dependence of scattering cross section on particle size, where the slope of line 30 is the value of the corresponding P3(i).

Prior to the experimental demonstration, colleagues expressed doubt regarding the practicality of the teachings herein for a number of reasons including:
the calculated value for P3(i) does not relate to any real physical property of tissue;
different wavelengths λ1 and λ2 penetrate to different depths in biological tissue so that the mechanism of scattering for two different λ1 and λ2 is substantially different;
absorbance of light by the tissue at λ1 and λ2 will introduce substantial errors, rendering calculated values for P3(i) useless; and
even if the value of P3(i) is related to Rayleign-Mie scattering, the expected differences in values of different P3(i) are too small to provide useful diagnostic information.

### Method of Generating an Image

Thus, according to an aspect of some embodiments of the teachings herein, there is provided a first method for generating an image of the surface of biological tissue, comprising:
a) receiving a first pixelated monochromatic image of an area of interest of the surface acquired during illumination of the surface with a first wavelength of light λ1;
b) receiving a second pixelated monochromatic image of the area of interest of the surface acquired during illumination of the surface with a second wavelength of light λ2, λ2 being different from λ1; and
c) generating a monochromatic third pixelated image from the first image and the second image by:
   for each desired location i of the area of interest, identifying a corresponding pixel P1(i) in the first image and a corresponding pixel P2(i) in the second image; and
   calculating a value for pixel P3(i) in the third image corresponding to the location i from a value of P1(i) and a value of P2(i), the value for P3(i) indicative of the relative amplitude scattering coefficient of tissue underlying the surface at the location i.

In some embodiments, the first method is a real-time method, that is to say, the acquired first and second images are received in real time and the third image is generated therefrom in real time. Alternatively, in some embodiments, the method is not a real-time method and the first and second images are recovered from storage.

According to an aspect of some embodiments of the teachings herein, there is also provided a method for generating an image of the surface of biological tissue, comprising:
a) acquiring a first pixelated monochromatic image of an area of interest during illumination of the surface with a first wavelength of light λ1;
b) acquiring a second pixelated monochromatic image of the area of interest during illumination of the surface with a second wavelength of light λ2, λ2 being different from λ1; and
c) receiving the first and second images and generating a monochromatic third pixelated image from the first image and the second image by:
   for each desired location i of the area of interest, identifying a corresponding pixel P1(i) in the first image and a corresponding pixel P2(i) in the second image; and
   calculating a value for pixel P3(i) in the third image corresponding to the location i from a value of P1(i) and a value of P2(i), the value for P3(i) indicative of the relative amplitude scattering coefficient of tissue underlying the surface at the location i.

As is clear to a person having ordinary skill in the art, in the second method, acquisition of the first and second images is performed with a camera. In some embodiments of the second method, the camera acquiring the first image and the camera acquiring the second image is the same camera. Alternatively, in some such embodiments, the camera acquiring the first image and the camera acquiring the second image are different cameras. In some embodiments, acquiring the first image and acquiring the second image are simultaneous. In some embodiments, acquiring the first image and acquiring the second image are not simultaneous. In some embodiments, the second method is a real-time method, where the acquiring and receiving of the first and second images and subsequent generation of the third-image therefrom is in real time.

As is clear to a person having ordinary skill in the art, in the first and second methods, the third image is received by and generated from the first and second images using one or more computer processors together with the required additional hardware and software such as power supplies, busses, digital memories, input ports, output ports, peripherals, operating systems and drivers. The methods can be implemented using any suitable computer processor for instance, using one or more custom processors and/or one or more commercially-available processors configured for implementing the methods using software and/or firmware and/or hardware.

In some embodiments, P3(i) is an approximation of the value of the slope of Rayleigh-Mie scattering cross section as a function of wavelength. In some embodiments, P3(i) is a linear approximation of the value of the slope of Rayleigh-Mie scattering cross section as a function of wavelength.

In some embodiments, P3(i) is related to the ratio ΔP(i) to Δλ, where:
ΔP(i) is the difference between the values of P1(i) and P2(i); and
Δλ is the difference between λ1 and λ2.

In some embodiments, wherein P3(i) is calculated using the formula:

(P1(i)−P2(i))/(λ1−λ2)

or substantially-equivalent formula as discussed hereinabove, so that P3(i) is a linear approximation of the value of the slope of Rayleigh-Mie scattering cross section as a function of wavelength.

In some embodiments, subsequent to ‘c’ the methods further comprise at least one of:
outputting the third image to a display component and the display component producing an image visible to a human from the third image;
outputting the third image to a storage component and the storage component storing the third image for future access; and
further processing a third image to generate useful information and outputting the useful information to a display component and/or to a storage component.

Any suitable display component may be used. In some embodiments, the display component is selected from the group consisting of a tangible display generator (e.g., a printer, a plotter) and a transient display (electronic display screen, LCD screen, LED screen).

Any suitable storage component may be used. In some embodiments the storage component is selected from the group consisting of hard disk, flash memory and cloud storage.

Any suitable processing method may be used to generate any useful information, typically clinically-useful information. In some embodiments, the processing method comprises or consists of generating a histogram of some or all of a third image. In some embodiments, the processing method comprises or consists of generating a Fourier Transform of some or all of a third image. In some embodiments, the processing of the third image includes identifying anomalous features in the third image as the useful information. In some embodiments, the processing of the third image comprises comparing the third image to a reference image of the area of interest, e.g., to identify a change which is considered an anomalous feature.

The methods of the teachings herein may be used to generate an image of the surface of any suitable biological tissue. In some embodiments, the surface is a biological surface selected from the group consisting of a brain, skin, mucosa, gastrointestinal mucosa, oral mucosa, a gynecological tract surface, and a respiratory tract surface.

### Properties of First and Second Images

Any suitable pair of first and second monochromatic pixelated image may be used in implementing the teachings herein.

The spatial resolution of the first and second images (the physical dimensions of a location that is represented by a single pixel) is any suitable spatial resolution. In some embodiments each pixel of the first and second image represents an area of biological tissue that is not more than 1 mm² (1×10⁶ micrometer²) and in some embodiments not more than 1×10⁵ micrometer² and even not more than 1×10⁴ micrometer². In some embodiments, each pixel of the first and second image represents an area of biological tissue that is not less than 1 micrometer².

The digital resolution of the first and second images (number of pixels that correspond to the area of interest of the surface) is any suitable digital resolution and, in preferred embodiments is identical. In some embodiments, the digital resolution is not less than 0.5 kP (kilopixels), not less than 1 kP, not less than 5 kP and even not less than 10 kP. In some embodiments, the digital resolution is not greater than 10⁸ MP.

The dynamic range (the number of discrete values each pixel can have) of the first and second images is any suitable dynamic range. In some embodiments, the dynamic range is not less than 16, not less than 32, not less than 64 and even not less than 128. In some embodiments, the dynamic range is not greater than 10⁸.

### Illumination and Image Acquisition

Some methods according to the teachings herein include receiving already-acquired first and second images. Some methods according to the teachings herein include acquiring first and second images.

In some embodiments, the first and second images are video images, that is to say, are images that are part of a set of images that together make up a video. In some embodiments, the first and second images are still images. In some embodiments, the first and second images are still images extracted from a video.

Whether or not acquisition is part of the invention, the first and second image are acquired with a camera, the first image during illumination with a first wavelength of light λ1 and the second image during illumination with a second wavelength of light λ2. Illumination of the surface of which the images are acquired is not part of the first method according to the teachings herein. Illumination of the surface of which the images are acquired with the required light is part of some embodiments of the second method according to the teachings herein.

### Illumination

As noted above, the first image is acquired with a camera during illumination with a first wavelength of light λ1 and the second image is acquired during illumination with a second wavelength of light λ2. In some embodiments, the second method further comprises: during the acquiring of the first image, illuminating the surface with a first wavelength of light λ1; and during the acquiring of the second image, illuminating the surface with a second wavelength of light λ2.

In some such embodiments, the illuminating of the surface with a first wavelength of light λ1; and the illuminating of the surface with the second wavelength of light λ2 are simultaneous. In some such embodiments, the illuminating of the surface with a first wavelength of light λ1; and the illuminating of the surface with the second wavelength of light λ2 are not-simultaneous.

λ1 and λ2

λ1 and λ2 are any two suitable wavelengths of light between 350 nm and 900 nm, and preferably between 395 nm and 645 nm. Due to considerations of the wavelength dependence of the relative importance of absorption to scattering of biological tissue (both oxy- and deoxyhemoglobin have substantial absorption peaks at around 400 nm) and the greater dependence of the slope of scattering as a function of wavelength at lower wavelengths as seen in FIG. 1, in some preferred embodiments λ1 and λ2 are any two suitable wavelengths of light between 420 nm and 600 nm, and even more preferably between 450 nm and 550 nm. For example, in some embodiments, λ1 is between 450 nm and 490 nm (e.g., 470 nm) and λ2 is between 510 nm and 550 nm (e.g., 530 nm).

Preferably, the illumination with light and the image acquisition with a camera are such that the first image is acquired from narrowband light with the first wavelength of light λ1 and the second image is acquired from narrowband light with the second wavelength of light λ2, both being not more than 30 nm FWHM (full-width at half-maximum). Preferably, both the first image and the second image are acquired from narrowband light having not more than 20 nm FWHM, not more than 10 nm FWHM and even not more than 5 nm FWHM.

In order to acquire the first and second image with narrowband light, narrowband illumination and/or narrowband acquisition are used. In some embodiments, the first and second images are acquired using narrowband illumination without narrowband acquisition. In some embodiments, the first and second images are acquired using narrowband illumination together with narrowband acquisition. In some embodiments, the first and second images are acquired using narrowband acquisition without narrowband illumination.

In narrowband illumination, a narrowband illuminator is used for illumination of the surface during acquisition of an image with narrowband light having not more than 30 nm FWHM, not more than 20 nm FWHM, not more than 10 nm FWHM and even not more than 5 nm FWHM, In some embodiments, a narrowband illuminator comprises a narrowband optical filter so that whatever physical component produces the illumination light, the light passes through the narrowband optical filter ensuring that the surface is illuminated by narrowband light. In some embodiments, a narrowband illuminator comprises a narrowband light source that produces narrowband light for illumination of the surface. Any suitable narrowband light source that that produces narrowband light can be used, including LEDs (light-emitting diodes) and lasers. In some embodiments, a LED light source is preferred to a laser light source as illumination with a laser can lead to speckled images and laser light may be considered too intense for safe illumination of tissue. In some embodiments, a narrowband illuminator comprises a narrowband light source such as a LED or laser functionally associated with a narrowband optical filter so that light produced by the narrowband light source passes through the narrowband optical filter before illuminating the surface.

In narrowband acquisition, the light used by a camera to acquire the first and second images is narrowband light having not more than 30 nm FWHM, not more than 20 nm FWHM, not more than 10 nm FWHM and even not more than 5 nm FWHM, In some embodiments, the camera is a spectral imaging camera that is configured to acquire narrowband images at different wavelengths. In some embodiments, the images are acquired using a camera that is functionally associated with a wavelength-selecting optical component is used so that during acquisition of the first image only narrowband light having a wavelength λ1 is acquired by the camera and during acquisition of the second image only narrowband light having a wavelength λ2 is acquired by the camera. Any suitable wavelength-selecting optical component or combination of different such optical components can be used including optical filters, prisms, diffraction gratings.

In some embodiments, illumination with the first wavelength of light λ1 and illumination of the surface with the second wavelength of light λ2 is simultaneous and in some embodiments is not-simultaneous. Further, in some embodiments acquisition of the first image and acquisition of the second image is simultaneous and in some embodiments is not-simultaneous. As is known to a person having ordinary skill in the art is able to select any combination of simultaneous/not-simultaneous illumination and simultaneous/not-simultaneous image acquisition using a required combination of narrowband illumination or not-narrowband illumination with narrowband acquisition or not-narrowband acquisition.

In some embodiments, the methods according to the teachings herein are implemented together with an additional imaging method or in a device already configured for implementing an additional imaging method. For example, in some embodiments a device is provided that includes one or more light sources that are suitable for illuminating a surface in a manner suitable for acquiring one or both of the first image and the second image. It is advantageous to implement the teachings herein with such methods and/or devices as these can be used together with the teachings herein to concurrently provide multiple different diagnostic images of the same area of interest.

This, in some embodiments, the teachings herein are integrated into an imaging device or configured to work together with an imaging device that can perform diagnosis with other imaging methods such as one or more of pulse oximetry, laser speckle contrast imaging (LCSI) and Blue Light Imaging (BLI).

In some embodiments, a method according to the teachings herein is implemented together with pulse oximetry. Pulse oximetry is known for use for determining oxygen saturation (SpO2) using two wavelengths of light: green (520-560 nm, especially 525 or 530 nm) with red (e.g., 600-750 nm, preferably 660-700 nm, especially 660 nm); or red (e.g., 600-750 nm, preferably 660-700 nm, especially 660 nm) with infrared (e.g., 850-100 nm, preferably 940 nm). In some such embodiments, λ1 is the green light used for implementing pulse oximetry and λ2 is a different, higher, wavelength of light. In some such embodiments, λ1 is the green light used for implementing pulse oximetry and λ2 is the red light used for implementing the pulse oximetry. In some such embodiments, λ2 is the green light used for implementing pulse oximetry and λ1 is a different, lower, wavelength of light (e.g., blue light 380-519 nm). In some such embodiments, λ2 is the red light used for implementing pulse oximetry and λ1 is a different, lower, wavelength of light (e.g., 380-599 nm).

In some embodiments, a method according to the teachings herein is implemented together with LCSI. LCSI is known using a red/NIR laser for illumination at 600-850 nm (especially 810 nm), e.g., for providing 2D perfusion maps of biological surfaces, such as of the gastrointestinal (GI) tract In some such embodiments, λ2 is the red/NIR light used for implementing LCSI and λ1 is a different, lower, wavelength of light (e.g., 380-599 nm). In some such embodiments, an embodiment of the method according to the teaching herein is implemented together with LCSI and with pulse oximetry.

In some embodiments, a method according to the teachings herein is implemented together with BLI. BLI is known using blue-light illumination, e.g., 410-450 nm, e.g., for the endoscopic characterization of mucosal changes in the GI tract. In some such embodiments, λ1 is the blue light used for implementing BLI and λ2 is a different, higher, wavelength of light. In some such embodiments, an embodiment of the method according to the teaching herein is implemented together with BLI and with LCSI, In some such embodiments, an embodiment of the method according to the teaching herein is implemented together with BLI and with pulse oximetry, In some such embodiments, an embodiment of the method according to the teaching herein is implemented together with BLI, LCSI and with pulse oximetry.

### Illumination and Acquisition for Performing the Second Method

As noted above, the second method according to the teachings herein comprises acquiring the first and the second image, in some embodiments simultaneously and in some embodiments not-simultaneously. Further, some embodiments of the second method according to the teachings herein comprise illuminating the surface with a first wavelength of light and with a second wavelength of light, in some embodiments simultaneously and in some embodiments not-simultaneously.

### Simultaneous Acquisition

In some embodiments of the second method, the first and second images are acquired simultaneously.

In such embodiments, the area of the interest is illuminated simultaneously with light having a wavelength λ1 and light having a wavelength λ2. In some such embodiments, the area of interest is illuminated with a continuous-wavelength light source such as white light or a xenon lamp which includes wavelengths of light in addition to λ1 and λ2. In some such embodiments, area of interest is illuminated with at least two discrete light sources, one for illuminating the area of interest with light having a wavelength of λ1 and one for illuminating the area of interest with light having a wavelength of λ2.

In such embodiments of the second method, acquisition of the first and second images is performed by any suitable camera or combination of suitable cameras.

In some embodiments of the second method, acquisition of both the first and second images is with a spectral imaging camera (e.g., from Ximea GmbH, Munster, Germany) and the first and second images are each extracted from a single acquired multispectral image.

In some embodiments of the second method, acquisition of both the first and second images is with a color (e.g., RBG) camera and the first and second images are each extracted from a single acquired color image.

In some embodiments of the second method, each one of the first and second image is acquired using a separate camera (preferably a monochromatic camera). Such embodiments typically include a lens that directs light coming from the area of interest to a beam splitter. The beam splitter directs a first portion of the light through a component such as a filter configured to direct only light having a wavelength of λ1 to a first (preferably monochromatic) camera for acquiring the first image and a second portion of the light through a component such as a filter configured to direct only light having a wavelength of λ2 to a second (preferably monochromatic) camera for acquiring the second image.

### Non-Simultaneous Acquisition

In some embodiments of the second method, the first and second images are acquired non-simultaneously. In such embodiments, it is preferred to acquire the two images as quickly as possible one after the other to make identification of corresponding pixels easy especially when acquiring images of moving biological tissue and/or from a moving platform (e.g., a moving endoscope). In some embodiments, the two images are non-simultaneously acquired within 1 second one of the other, within ¼, within ⅛ seconds, within 1/16 and even within 1/32 of a second of each other.

### Simultaneous Illumination, Non-Simultaneous Acquisition

In some embodiments of the second method, the area of the interest is illuminated simultaneously with light having a wavelength λ1 and light having a wavelength λ2. Such simultaneous illumination may be according to the options described above, and is not repeated for brevity. In such embodiments, acquisition of the first and second images is performed by any suitable camera or combination of suitable cameras.

In some such embodiments, acquisition of both the first and second images is with the same or different spectral imaging or color (e.g., RGB) camera and the first and second images are each extracted from a single acquired multispectral or color image.

In some such embodiments, each one of the first and second image is acquired using a separate (preferably monochromatic) camera as described above.

In some such embodiments, both the first and second image are acquired using the same (preferably monochromatic) camera. Such embodiments typically include a lens that directs light coming from the area of interest to a changing wavelength director. The changing wavelength assembly is configured to direct light from the lens to the camera through a component that is controllably alternated between directing only light having a wavelength of λ1 to the camera for acquiring the first image and directing only light having a wavelength of λ2 to the camera for acquiring the second image. Suitable non-limiting examples of changing wavelength director include changing wavelength directors that comprise one or more of a filter wheel, a prisms and a diffraction grating.

### Device for Generating an Image

The methods according to the teachings herein may be implemented using any suitable device or combination of devices. In some preferred embodiments, a device according to the teachings herein is used for implementing the teachings herein.

According to an aspect of some embodiments of the teachings herein, there is provided a device for generating an image of the surface of biological tissue, comprising a computer processor having at least one input port and at least one output port, the computer processor configured to:
a) receive a first pixelated monochromatic image of an area of interest of the surface acquired during illumination of the surface with a first wavelength of light λ1 through the at least one input port;
b) receive a second pixelated monochromatic image of the area of interest of the surface acquired during illumination of the surface with a second wavelength of light λ2, λ2 being different from λ1 through the at least one input port; and
c) generate a monochromatic third pixelated image from the first image and the second image by:
   for each desired location i of the area of interest, identifying a corresponding pixel P1(i) in the first image and a corresponding pixel P2(i) in the second image; and
   calculating a value for pixel P3(i) in the image corresponding to the location i from a value of P1(i) and a value of P2(i), the value for P3(i) indicative of the relative amplitude scattering coefficient of tissue underlying the surface at the location i.

In some embodiments, at least one of:
the device further comprises a display component that is functionally associated with the at least one output port, the processor is further configured to generated a third image through the at least one output port, and the display component is configured to produce an image visible to a human from the third image;
the device further comprises a storage component that is functionally associated with the at least one output port, the processor is further configured to output the third image through the at least one output port, and the storage component is configured to store the third image received from the computer processor for future access;
the computer processor is further configured to process the third image to generate useful information and to output the useful information to a display component and/or a storage component through the output port.

In some embodiments, the device further comprises:
an illuminator for illuminating a surface with a first wavelength of light λ1 and with a second wavelength of light λ2;
a camera for acquiring a first image of an area of interest of a surface during illumination with the first wavelength of light λ1 by the illuminator and for providing an acquired first image to the computer processor through an input port; and
a camera for acquiring a second image of an area of interest of a surface during illumination with the second wavelength of light λ2 by the illuminator and for providing an acquired second image to the computer processor through an input port.

In some embodiments, the camera for acquiring the first image and the camera for acquiring the second image are the same camera. In some such embodiments, the device is configured so that the camera acquires a first image and a second image simultaneously. Alternatively, in some such embodiments, the device is configured so that the camera acquires a first image and a second image not-simultaneously.

In some embodiments, the camera for acquiring the first image is a first camera that is different from a second camera that is the camera for acquiring the second image. In some such embodiments, the device is configured so that the first camera acquires a first image and the second camera acquires a second image simultaneously. Alternatively, in some such embodiments, the device is configured so that the first camera acquires a first image and the second camera acquires a second image not-simultaneously.

In some embodiments, the camera for acquiring the first image and the camera for acquiring the second image (whether being the same camera or two different cameras) are configured to acquire an image such that each the pixel represents an area of biological tissue that is not more than 1×10⁶ micrometer².

In some embodiments, the camera for acquiring the first image and the camera for acquiring the second image (whether being the same camera or two different cameras) are configured to acquire an image such that each the pixel represents an area of biological tissue that is not less than 1 micrometer².

In some embodiments, the camera for acquiring the first image and the camera for acquiring the second image (whether being the same camera or two different cameras) are configured to acquire an image such that each the pixel has a dynamic range that is not less than 16.

In some embodiments, the camera for acquiring the first image and the camera for acquiring the second image (whether being the same camera or two different cameras) are configured for narrowband acquisition of light having not more than 30 nm FWHM and preferably not more than 20 nm FWHM, not more than 10 nm FWHM and even not more than 5 nm FWHM. In some such embodiments, the configuration for narrowband acquisition comprises functional-association of the camera with a wavelength-selecting optical component. Any suitable wavelength-selecting optical component or combination of components can be used, in some such embodiments being the wavelength-selecting component is at least one component selected from the group consisting of an optical filter, a prism and a diffraction grating.

In some embodiments, the illuminator is configured to illuminate a surface with the first wavelength of light λ1 and with the second wavelength of light λ2 simultaneously.

In some embodiments, the illuminator is configured to illuminate a surface with the first wavelength of light λ1 and with the second wavelength of light λ2 not-simultaneously.

In some embodiments, the illuminator is configured for illuminating a surface with: narrowband light with the first wavelength of light λ1; and narrowband light with the second wavelength of light λ2, both narrowband light having not more than 30 nm FWHM and preferably not more than 20 nm FWHM, not more than 10 nm FWHM and even not more than 5 nm FWHM. In some embodiments, the configuration for illuminating a surface with narrowband light comprises functional-association of a light source with a wavelength-selecting optical component. Any suitable wavelength-selecting optical component or combination of components can be used, in some such embodiments being the wavelength-selecting component is at least one component selected from the group consisting of an optical filter, a prism and a diffraction grating. In some such embodiments, the configuration for illuminating surface with narrowband light with the first wavelength of light λ1 comprises a first narrowband light source producing the narrowband light with the first wavelength of light λ1; and the configuration for illuminating surface with narrowband light with the second wavelength of light λ2 comprises a second narrowband light source producing the narrowband light with the second wavelength of light λ2. In some such embodiments, the first and the second narrow band light sources are selected from the group consisting of a LED and a laser.

In some embodiments, the device is an endoscope, in some embodiments an endoscope selected from the group consisting of arthroscope, bronchoscope, colonoscope, cystoscope, duodenoscope, enteroscope, gastroscope, hysteroscope, laparoscope, laryngoscop, nephroscope and ureteroscope.

As discussed above with reference to the methods according to the teachings herein, In some embodiments, the device is configured to perform at least one additional medical imaging method, in some such embodiments the at least one additional medical imaging method is selected from the group consisting of pulse oximetry, LCSI and BLI.

In various embodiments of the devices variations and options such as the values for pixel P3(i), image properties, illumination properties and image acquisition properties are as described hereinabove for the embodiments of the first and second methods according to the teachings herein. These embodiments and variations are not repeated for the sake of brevity but are understood to be disclosed and provide literal support for such embodiments and variations as if explicitly listed here.

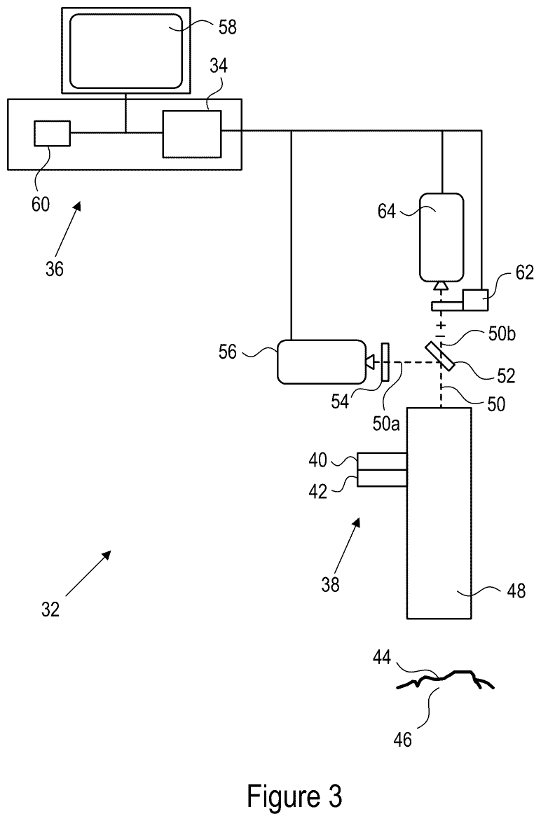

An embodiment of a device according to the teachings herein, device 32, a gastrointenstinal endoscope, is schematically depicted in FIG. 3. Device 32 comprises a computer processor 34 including an input port and an output port. Computer processor 34 is a component of a commercially-available general purpose computer 36 that includes all the required hardware and software for implementation of the teachings herein. Computer processor 34 is software-configured using commercially-available software such as Matlab® (by Mathworks, Natick, MA, USA) or Python® (by Python Software Foundation, DE, USA) to receive acquired first and second images and to generate a third image therefrom in accordance with the teachings herein.

Device 32 comprises an illuminator 38 including two light sources, 810 nm laser 40 and broadband light source 42 (a LED from LCFOCUS-HP LED-FS5-03, full spectrum sunlight light source suitable for indoor plant growing producing light from 380 nm to 840 nm, China) which are configured to allow simultaneous illumination of a surface 44 of tissue 46 with 810 nm laser light generated by laser 40 and white light from light source 42 through endoscope body 48.

Light 50 returned from tissue 46 is directed by endoscope body 48 to beam splitter 52 that splits returned light 50 to beam 50a and beam 50b.

Beam 50a is directed to pass through optical filter 54 that allows only light having wavelengths of 400 nm to 700 nm to pass therethrough to RGB video camera 56. RGB video camera 56 acquires RGB video images and provides these to processor 34. Processor 34 continuously displays on screen 58 and stores on hard disk 60 the acquired RGB color images as video so that medical personnel can use device 32 as an endoscope in the usual way, observing RGB video of an area of interest on surface 44 of tissue 46 on screen 58.

Beam 50b is directed to pass through towards variable optical filter 62. Variable optical filter 62 is a motorized variable-speed optical wheel (e.g., from Zaber Technologies, Vancouver, BC, Canada) bearing four optical filters: i. a narrowband 470 nm optical filter with 5 nm FWHM; ii. a narrowband 530 nm optical filter with 5 nm FWHM; iii. a narrowband 660 nm optical filter with 5 nm FWHM; and iv. a narrowband 810 nm optical filter with 5 nm FWHM.

After passing through variable optical filter 62, beam 50b is acquired by 120 fps monochrome CCD video camera 64. The rotation of variable optical filter 62 and the image acquisition of camera 64 are coordinated so that camera 64 acquires 120 images every second: 30 first images acquired at a λ1 of 470 nm, 30 second images acquired at a λ2 of 530 nm, 30 images acquired at 660 nm and 30 images acquired at 810 nm. The images acquired by camera 64 are provided to and received by processor 34.

From each pair of a first image (acquired at λ1 of 470 nm) and second image (acquired at λ2 of 530 nm) acquired with approximately 0.01 second time difference, processor 34 generates a third image in accordance with the teachings herein in real time at a rate of 30 third images every second. An operator can optionally use processor 34 to calculate histograms of groups of pixels from the generated third images and display the histograms on screen 56. For instance, an operator can define 30×30 pixel square tiles of the third images as groups and use processor 34 to calculate the histogram of each group of pixels.

Processor 34 further generates a series of LCSI images that depict blood flow in the usual way from the images acquired at 810 nm.

Processor 34 further generates a series of oxygen saturation (SpO2) in the usual way known from pulse oximetry from the images acquired at 530 nm and 660 nm.

Processor 34 further generates a series of metabolic rate of oxygen (MRO2) from the LCSI and SpO2 images in the usual way.

As a result, processor 34 generates six diagnostic video image series of surface 44: RGB images, third images, histogram of third images, LCSI images, SpO2 images and MRO2 images. Processor 34 continuously stores all six video image series on hard disk 60 and displays one, two, three, four, five or six of the videos on screen 58, each in a separate tile, as desired by an operator. Further, using the standard functions found in Wolfram Mathematica, an operator can choose to display a fused or coregisted composite of the RGB images with one of the other images.

### EXAMPLE

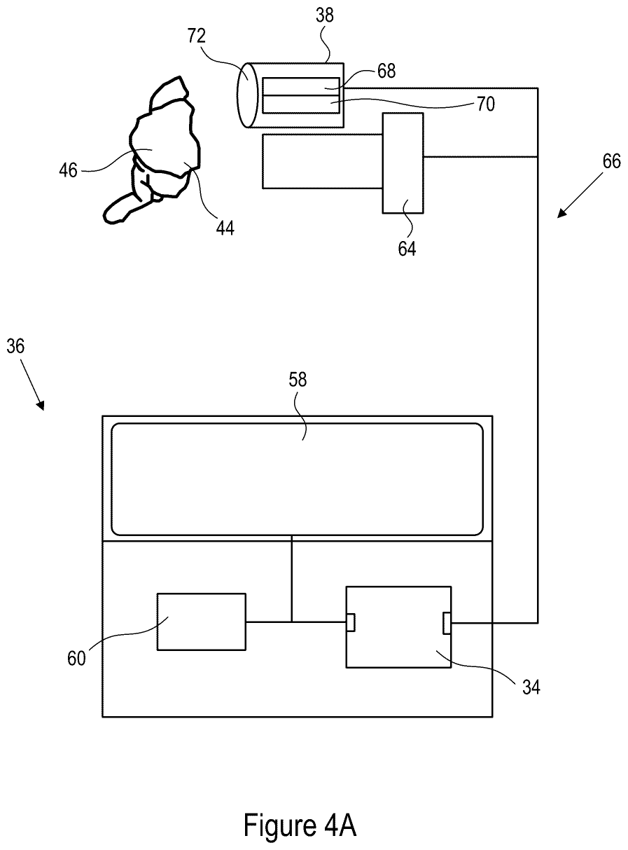

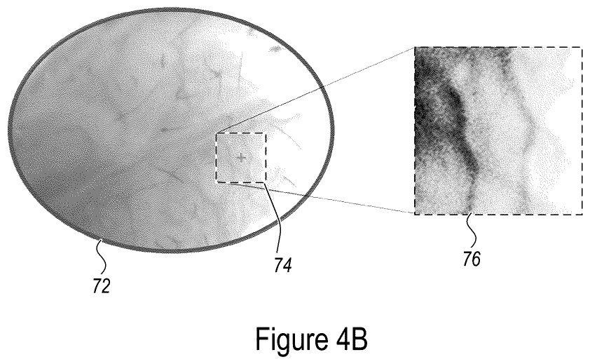

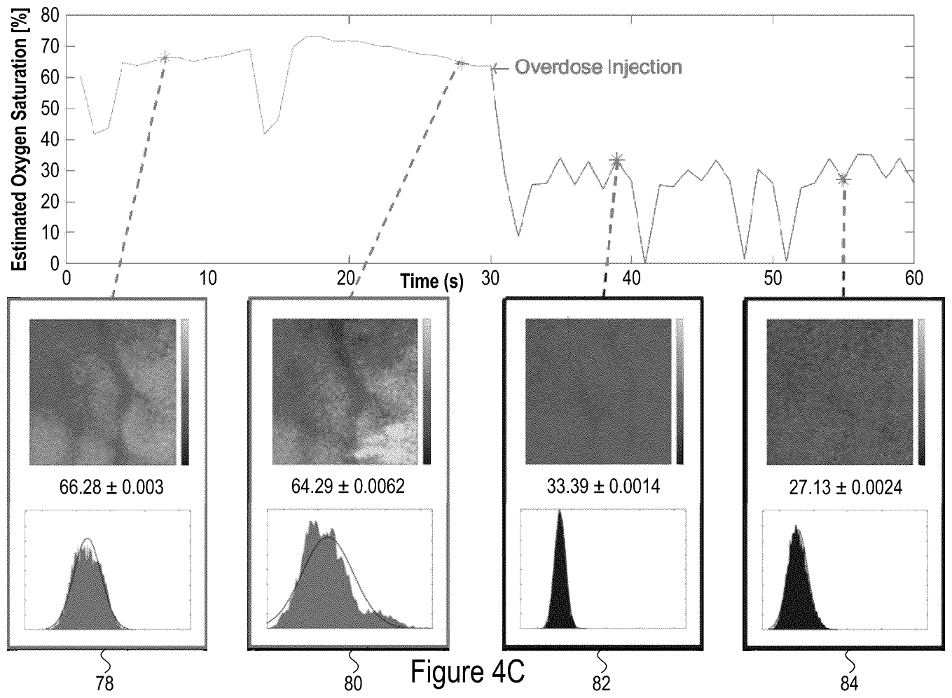

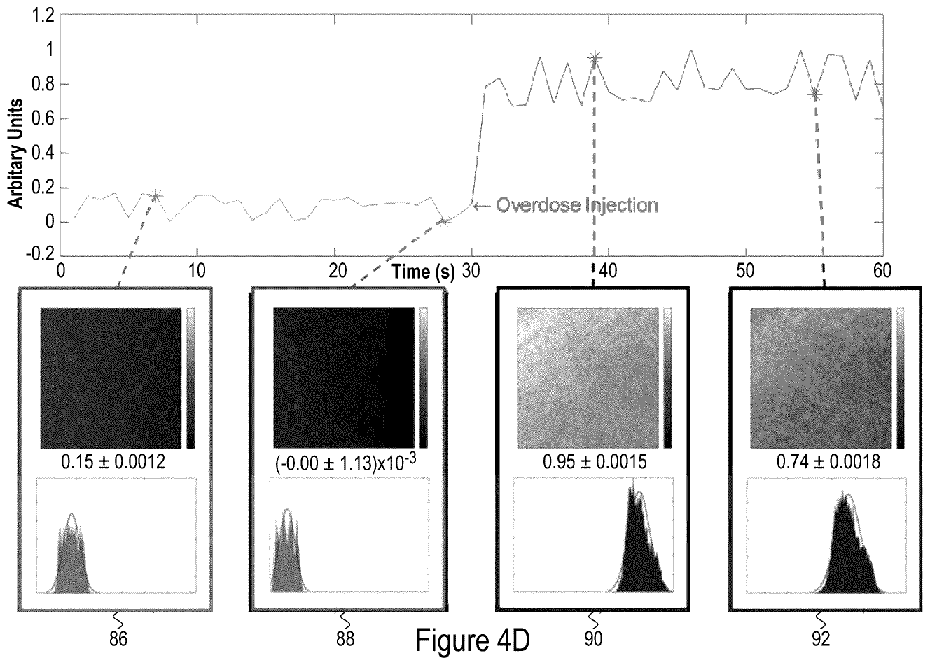

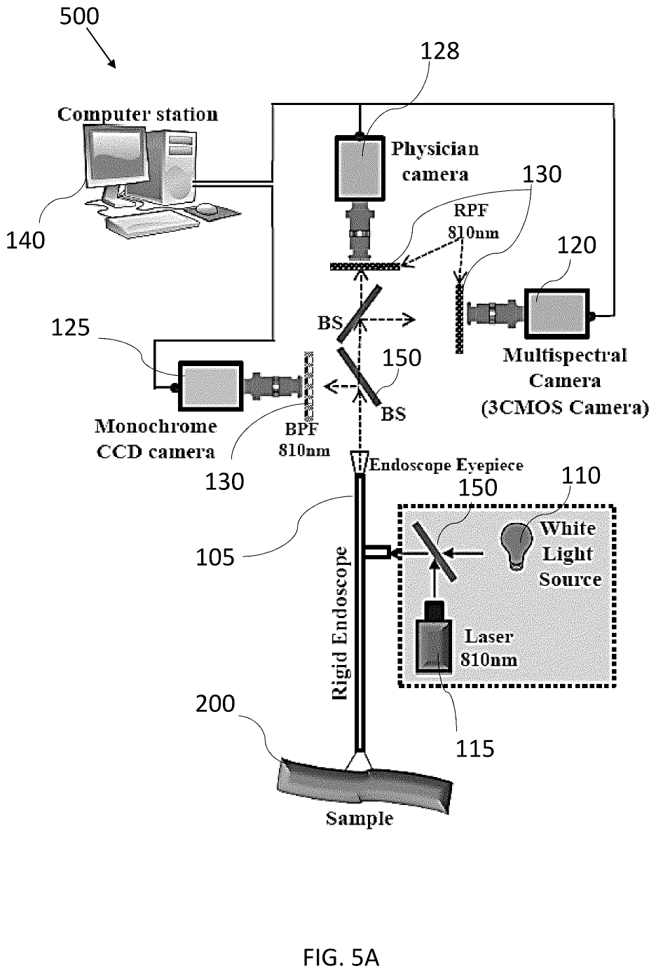

An experiment performed to demonstrate the teachings herein is described below with reference to FIGS. 4A-4D

### Experimental Device

An embodiment of a device according to the teachings herein, device 66, was made and is schematically depicted in FIG. 4A.

Device 66 include a commercially-available general-purpose laptop 36 including a computer processor 34, configured with the required software programs and drivers to control other components of the device and to process data in accordance with the teachings herein. Image acquisition, synchronization and data analysis were performed using scripts written by the Inventors and/or their assistants using Matlab®.

Device 66 included an illuminator 38 comprising a first laser 68 generating green light having a wavelength λ1=532 nm (3 FWHM) and a second laser 70 generating red light having a wavelength λ2=660 nm (3 FWHM), lasers 68 and 70 independently-operable by computer 36 and processor 34. Both lasers directed generated light through a positive lens 72 to create a collimated beam of light having a diameter of −15 mm.

Device 66 included a monochrome CCD camera 64 (GuppyPro F-031B by Allied Vision Technologies GmbH, Stadtroda, Germany, configured to acquire monochrome images having a digital resolution of 0.3 MB (656×492) at a rate of 123 fps) equipped with a macro zoom lens (MLH10X F5.6-32 by Computar, Chuo-ku, Tokyo 104-0052, Japan).

Computer 36 and processor 34 were configured to:
to alternatingly activate one of the two lasers 68 and 70 in quick succession to illuminate a surface 44 of biological tissue 46 through positive lens 72;
during activation of a laser 68 or 70, to activate camera 64 to acquire an image of illuminated surface 44;
to receive an acquired image from camera 64 and store the received image as either a first image or a second image of the teachings herein;
when desired (typically when illuminator 38 and camera 64 were not active), to generate a diagnostic third image from a first image and corresponding second image in accordance with the teachings herein; to store a generated third image on a hard disk 60 of computer 36; to display a generated third image on a screen 58 of computer 36; and to calculate the mean pixel value in a given third image.

### Experiment

A mouse was sedated in the usual way and then immobilized. A portion of the brain of the mouse was surgically exposed and the exposed portion placed so that the image-acquisition module could continuously acquire images of the exposed brain. FIG. 4B is a reproduction of an image of the mouse brain acquired by the image-acquisition module. The reproduced image in oval 72 is the entire field-of-view of camera 64 while the area delineated by the dotted lines 74 and displayed in enlarged form 76 is the area of interest.

Camera 64 was activated to acquire images at 120 fps in coordination with alternating activation of the two lasers so that every second camera 64 acquired 60 first images during illumination with λ1=532 nm and 60 second images during illumination with λ2=660 nm. The acquired images were stored in hard disk 60.

While camera 64 was acquiring images of the brain, the mouse was given an overdose of anaesthesia leading to a quick and painless death.

After the experiment was complete, a monochromatic pixelated cerebral tissue oxygenation (SpO2) image was generated for each first/second image pair as known in the art of pulse oximetry using λ1=532 nm (green) and λ2=660 nm (red). Specifically, the value of each pixel in the generated oxygenation image was calculate from the value of the corresponding pixels in the first (green) image and the second (red) image. For each oxygenation image, a single mean value of all the pixels was calculated. The mean value expressed the average oxygenation value of the entire area of interest at the moment the image pair was acquired, higher values indicating greater oxygenation and lower values indicating less oxygenation. In FIG. 4C, the mean oxygenation values are plotted as a function of time to provide a graphic depiction of the time-dependent variation in cerebral tissue oxygenation caused by the anaesthesia overdose and consequent death. FIG. 4C also includes a reproduction of two oxygenation images and corresponding histograms 78 and 80 prior to the overdose and a reproduction of two oxygenation images and corresponding histograms 82 and 84 subsequent to the overdose.

A third image according to the teachings herein was generated for each first/second image pair as described herein. For each such third image, a single mean value of all the pixels was calculated. The mean value expressed the average scattering value of the entire area of interest at the moment the image pair was acquired. In FIG. 4D, the mean scattering values are plotted as a function of time to provide a graphic depiction of the time-dependent variation in scattering caused by the anaesthesia overdose and consequent death. It is believed that dramatic increase in scattering value relates to morphological changes that occur in the brain tissue as a result of cell swelling and/or apoptosis. FIG. 4D also includes a reproduction of two third-images and corresponding histograms 86 and 88 prior to the overdose and a reproduction of two third-images and corresponding histograms 90 and 92 subsequent to the overdose. From these four third-images showing the sensitivity of the method according to the teachings herein.

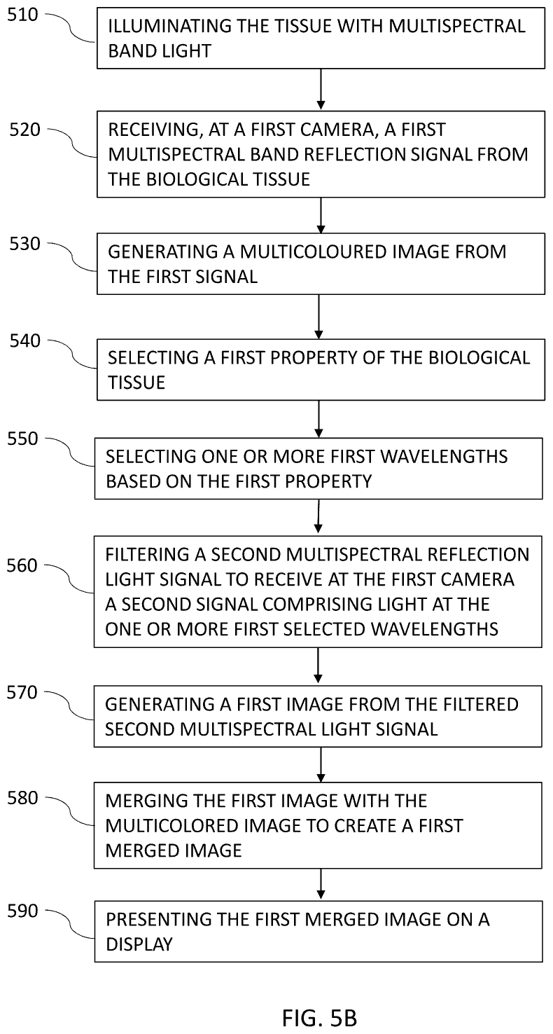

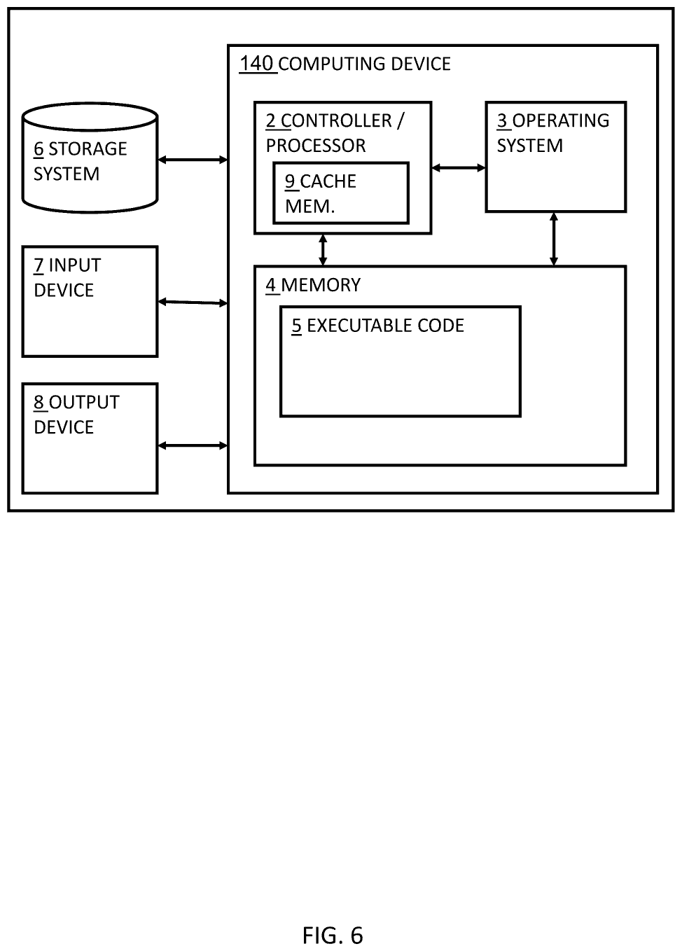

Reference is now made to FIG. 5A which is an illustration of a system for diagnosing a biological tissue according to some embodiments, of the invention. System 100 may include at least a multispectral light source 110 (e.g., a white light source), a multispectral camera 120 and a wavelength filter 130. Multispectral light source 110, multispectral camera 120 and wavelength filter 130 may all be optically connected with optical connection set 150 which may include any required number of mirrors, waveguides and lenses. Multispectral light source 110, multispectral camera 120 and wavelength filter 130 may further be optically connected to an endoscope 105 configured to be inserted into a patient's body. System 100 may further include a computing device 140, discussed herein below with respect to FIG. 6.

Multispectral light source 110 may be any light source configured to emit light at two or more different wave lengths, for example, a range of wavelengths. Multispectral light source 110 may be white light source, red light source (e.g., emitting light at 600-700 nm), green light source (e.g., emitting light at 520-560 nm) and the like. For example, multispectral light source 110 may be or may include broadband light source 42, discussed herein above.

Multispectral camera 120, may be any RGB camera, for example, RGB camera 56, discussed herein above.

In some embodiments, wavelength filter 130 may be an optical wavelength filter located in front of camera 120, as illustrated. Additionally or alternatively, wavelength filter 130 may be an a controller associated with camera 120, configured to select specific wavelengths from the total wavelengths captured by the camera and isolate images captured in these wavelengths. For example, optical filter 120 may be or may include optical filter 54 that allows only light having wavelengths of 400 nm to 700 nm to pass therethrough to RGB video camera 56. In yet another example, optical filter 120 may be or may include variable optical filter 62 which is a motorized variable-speed optical wheel bearing four optical filters, discussed herein above.

Endoscope 105 may be any endoscope, for example, the gastrointestinal endoscope body 48 discussed herein above with respect to device 32. In other examples, endoscope 105 may be laryngoscopy, colonoscope, cystoscope, gastroscope, laparoscope and the like.

In some embodiments, system 100 may further include a monochromatic light source 115, for example, a laser (e.g., the 810 nm laser illustrated) for providing illumination of the tissue at a single specific light. In some embodiments, multispectral light source 110 and monochromatic light source 115 may be configured to allow simultaneous illumination of a surface 44 of tissue 46 with 810 nm laser light generated by laser 40 and white light, as discussed with respect to light source 42 and laser 40, discussed herein above.

In some embodiments, system 100 may further include monochrome camera 125, configured to capture a single wavelength, for example, 810 nm. In some embodiments, monochrome camera 125 may be a 120 fps monochrome CCD video camera, such as, monochrome CCD video camera 64, discussed herein above. In some embodiments, another wavelength filter 130 may be placed in front of the lens of monochrome camera 125.

In some embodiments, system 100 may include or may be in communication with physician camera 128, which may be any broadband video camera, configured to provide the physician operating endoscope 105 an image of the tissue. In some embodiments, another wavelength filter 130 may be placed in front of the lens of physician camera 128 allowing physician camera 128 to capture only images at selected wavelengths.

In some embodiments, one or all of the components of system 100 may be controlled or may be in communication with controller 140.

Reference is now made to FIG. 5B which is a flowchart of a method of diagnosing a biological tissue according to some embodiments of the invention. The method of FIG. 5B may be performed by system 100 and/or device 32 using a computing device, such as, computing device 140 or by any suitable computing device and system.

In step 510, the biological tissue may be illuminated with multispectral band light. For example, computing device 140 may controller multispectral illumination source 110 to illuminate sample tissue 200, illustrated in FIG. 5A. The multispectral band light may be delivered via endoscope 105 to sample tissue 200. The multispectral band light may be a white light or any other light band, for example, red light source (e.g., emitting light at 600-700 nm), green light source (e.g., emitting light at 520-560 nm) and the like.

In step 520, a first multispectral band reflection signal from the tissue may be received at a first camera. For example, first multispectral band reflection signal may be received at multispectral camera 120. In some embodiments, the first multispectral band reflection signal may include the entire visible spectrum (e.g., white light) or may include narrower bands, for example, the red and the green bands 520-700 nm.

In step 530, multicolored image may be generated from the first signal. For example, computing device 140 may generate the multicolored image from the multispectral band reflection signal.

In step 540, a first property of the biological tissue may be selected. The first property may be indicative of a condition of the biological tissue and may further assist in diagnosing the tissue. For example, computing device 140 may receive from an external user device, or a user interface (e.g., via input devices 7) a selection (made by a user) of the property. Additionally or alternatively, computing device 140 may be preprogramed to select the property, for example, using a code stored in memory 4. Some nonlimiting examples for such properties may include, the tissue's oxidation level (e.g., saturation), glucose levels, a perfusion of biological surfaces level lipids amount, water level, metabolic rate of oxygen, hemoglobin, and the like. In some embodiments, detecting/monitoring the selected property may allow diagnosing a condition (e.g., a medical condition) of the biological tissue. Changes in the selected properties, with respect to healthy tissues, may be indicative of a medical problem.

In step 550, one or more first wavelengths may be selected based on the first property. For example, based on the selected property, computing device 140 may select one or more first wavelengths, using a lookup table stored in memory 4. In a first nonlimiting examples the first selected property is oxygen level, and the first one or more wavelengths include at least two wavelengths are selected from green light at 520-560 nm. In a second nonlimiting examples the first selected property is deoxyhemoglobin level, and the first one or more wavelengths is a single wavelength selected from red light at 600-700 nm. In a third nonlimiting examples the first selected property is perfusion of biological surfaces level, and the first one or more wavelengths is a single wavelength selected form 630-850 nm, for example, 630 nm, 780 nm, 810 nm and the like.

In step 560, a second multispectral reflection light signal may be filtered to receive at the first camera a second signal comprising light at the one or more first selected wavelengths. In some embodiments, a second multispectral reflection light signal, which is a result of the multispectral illumination of the tissue, in step 510, may be filtered using filter 130 prior to being acquired by camera 120. In such case filtering the one or more first wavelengths may be done by an optical filter (e.g., filters 54 and 62), placed in front of the camera. In some embodiments, filtering the one or more first wavelengths is by selecting the one or more wavelengths in the second multispectral signal.

In some embodiments, the second signal may be received by illuminating the tissue with a laser, for example, monochromatic source 115 and/or laser 40. Accordingly, the signal may initially include only a single selected wavelength, for example, 810 nm.

In step 570, a first image may be generated from the filtered second multispectral light signal. In some embodiments, computing device 140 may generate the first image. In some nonlimiting examples, the first image may be a monochromatic image (e.g., when the property is perfusion of biological surfaces level), a biochromatic image (e.g., when the property is oxygen level), a trichromatic image and the like.

In step 580, the first image may be merged with the multicolored image to create a first merged image, using any known overlaying/merging method. In step 590, the first merged image may be displayed on a display. For example, computing device 140 may merge the first image and the multicolored image and may display the first merged image on a display associated with computing device 140, for example, a display included in output device 8.

In some embodiments, the method may further include selecting a second property of the biological tissue. For example, if the first selected property was oxidation level, the second selected property may be perfusion of biological surfaces level. In some embodiments, a corresponding second wavelengths may be selected based on the second property, as discussed herein above with respect to steps 540 and 550. In some embodiments, a third multispectral reflection light signal may be filtered to receive at the first camera a second signal comprising light at the second one or more selected wavelengths. For example, the third multispectral reflection light signal may be filtered using filter 130 prior to being acquired by camera 120. In such case filtering the one or more second wavelengths may be done by an optical filter (e.g., filters 54 and 62), placed in front of the camera. In some embodiments, filtering, the one or more second wavelengths, is by selecting the one or more wavelengths in the third multispectral signal.

In some embodiments, a second image may be generated from the filtered third multispectral reflection light signal; may be merged the second image on the multicolored image to create a second merged image. The second merged image may be presented on a display. Alternatively, the method may include merging the second monochromatic image on the first combined image to create a third combined image; and presenting the third combined image on a display.

In some embodiments, the method may further include illuminating the tissue with monochromatic light, for example, using source 115 or laser 40. In some embodiments, a monochromatic signal, from the biological tissue, may be received at a monochrome camera (e.g., monochrome camera 125). In some embodiments, a monochromatic image may be generated from the monochromatic signal and may be merged with the multicolored image to form a fourth merged image. The fourth image may be displayed on a displayed.

In some embodiments, the method may include receiving, at a second camera (e.g., physician camera 128), a white light reflection signal from the biological tissue and generating and presenting a white light image, for example, on a display.

In some embodiments, the first merged image may be displayed on a first display, the second merged image may be displayed on a second display and/or the third merged image may be displayed on a third display and the like. In some embodiments, the first, second third and/or fourth merged images may be displayed on a single display (e.g., the same screen) one next to the other, optionally along the white light image.

Reference is now made to FIG. 6, which is a block diagram depicting a computing device, which may be included within an embodiment of a system for diagnosing a biological tissue, according to some embodiments.

Computing device 140 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 140 may be included in, and one or more computing devices 140 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 140, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task or script. Executable code 5 may be executed by processor or controller 2 possibly under control of operating system 3. For example, executable code 5 may be an application that may TBD as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data TBD may be stored in storage system 6 and may be loaded from storage system 6 into memory 4 where it may be processed by processor or controller 2. In some embodiments, some of the components shown in FIG. 6 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 140 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 140 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

As used herein, for clarity the term “image” refers to a visible image (e.g., as displayed on permanent media such as on printed paper or electronic media such as a display screen (LED, LCD, CRT)), as well as image data (especially electronic data) representing the image including data stored, for example, on magnetic or electrical media (e.g., RAM, flash memory, magnetic disk, magnetic tape).

As used herein, for clarity the term “pixel” refers to an element making up a pixelated image (displayed or stored as data) and also to the value of the pixel, as the context dictates.

As used herein, the term “monochrome image data” refers to digital data representing a pixelated image where the value of each pixel is a single intensity value representing only an amount of light, that is, it carries only intensity information.

As used herein, a computer processor is an electronic device that can be programmed to perform mathematical functions and data processing. Non-limiting examples of the term processor include microprocessors, digital signal processors (DSP), microcontrollers, field programmable gate arrays (FGPA), application specific integrated circuits (ASIC) as well as devices such as computers, personal computers, servers, smart phones and tablets. For implementing the teachings herein, such computer processors are typically programmed, e.g., through the use of software instructions to carry out the functions and methods described herein.

As used herein, the term “camera” refers to any device capable of generating a digital pixelated image data (as stills or video).

Unless otherwise defined, all technical and scientific terms used herein have the same meaning as commonly understood by one of ordinary skill in the art to which the invention pertains. In case of conflict, the specification, including definitions, will take precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

As used herein, when a numerical value is preceded by the term “about”, the term “about” is intended to indicate +/−10%.

As used herein, a phrase in the form “A and/or B” means a selection from the group consisting of (A), (B) or (A and B). As used herein, a phrase in the form “at least one of A, B and C” means a selection from the group consisting of (A), (B), (C), (A and B), (A and C), (B and C) or (A and B and C).

Embodiments of methods and/or devices described herein may involve performing or completing selected tasks manually, automatically, or a combination thereof. Some methods and/or devices described herein are implemented with the use of components that comprise hardware, software, firmware or combinations thereof. In some embodiments, some components are general-purpose components such as general purpose computers, digital processors or oscilloscopes. In some embodiments, some components are dedicated or custom components such as circuits, integrated circuits or software.

For example, in some embodiments, some of an embodiment is implemented as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more of input devices (e.g., allowing input of commands and/or parameters) and output devices (e.g., allowing reporting parameters of operation and results.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Citation or identification of any reference in this application shall not be construed as an admission that such reference is available as prior art to the invention.

Section headings are used herein to ease understanding of the specification and should not be construed as necessarily limiting.

1. A method for diagnosing a biological tissue, comprising:
a) illuminating said tissue with multispectral band light;
b) receiving, at a first camera, a first multispectral band reflection signal from said biological tissue;
c) generating a multicoloured image from said first signal;
d) illuminating said biological tissue with a monochromatic light;
e) receiving, at a second camera, a monochromatic signal from said biological tissue;
f) generating a monochromatic image from said monochromatic signal;
g) selecting a first property of said biological tissue;
h) selecting one or more first wavelengths based on said first property;
i) filtering a second multispectral reflection light signal to receive at said first camera, a second signal comprising light at said one or more first wavelengths;
j) generating a first image from said filtered second multispectral light signal;
k) merging said first image with said multicolored image to create a first merged image;
l) merging said multicolored image with said monochromatic image to form a second merged image; and
m) presenting said first merged image and the second merged image on a display.

2. The method of claim 1, wherein filtering the one or more first wavelengths is by an optical filter, placed in front of said first camera.

3. The method of claim 1, wherein filtering the one or more first wavelengths is by selecting said one or more wavelengths from said second multispectral light signal.

4. The method according of claim 1, wherein said first property is oxygen level, and the first one or more wavelengths include at least two wavelengths selected from green light at 520-560 nm.

5. The method according of claim 1, wherein said first property is deoxyhemoglobin level, and the first one or more wavelengths is at least one wavelength selected from red light at 600-700 nm.

6. The method according of claim 1, wherein said first property is perfusion of biological surfaces level, and the first one or more wavelengths is a single wavelength selected form 630-850 nm.

7. The method of claim 5, wherein said second signal is received by illuminating said biological tissue with a laser at the selected wavelength and generating the first image is generating a monochromatic image.

8. The method of claim 1, further comprising:
a) selecting a second property of the biological tissue;
b) selecting one or more second wavelengths based on said second property;
c) filtering a third multispectral reflection light signal to receive at the first camera a second signal comprising light at said second one or more selected wavelengths;
d) generating a second image from said third multispectral reflection light signal; and
e) merging said second image with the multicolored image to create a third merged image; and
f) presenting said third merged image on a display.

9. The method of claim 1, further comprising:
a) selecting a second property of the biological tissue;
b) selecting one or more second wavelengths based on said second property;
c) filtering said second multispectral reflection light signal to receive at said first camera also a second signal comprising light at the second one or more selected wavelengths; and
d) generating a second image from said second multispectral light signal;
e) merging said second monochromatic image with said first combined image to create a fourth combined image; and
f) presenting said fourth combined image on a display.

10. The method of claim 1, wherein said multispectral reflection light is white light.

11. (canceled)

12. The method of claim 11, wherein illuminating said tissue with monochromatic light is by a laser source.

13. The method of claim 1, further comprising:
receiving, at a second camera, a white light reflection signal from the biological tissue; and
generating and presenting a white light image.

14. A system for diagnosing a biological tissue, comprising:
at least one multispectral light source;
a monochromatic light source;
at least one first multispectral camera;
a second camera;
a filter configured to filter one or more wavelengths from a multispectral reflection light signal; and
and a controller configured to:
a) control said multispectral light source to illuminate said tissue with multispectral band light;
b) receive, from a first camera, a first multispectral band reflection signal from said biological tissue;
c) generate a multicoloured image from said first signal;
d) control said monochromatic light source to illuminate said biological tissue with a monochromatic light;
e) receive at said second camera, a monochromatic signal from said biological tissue;
f) generate a monochromatic image from said monochromatic signal;
g) select a first property of said biological tissue;
h) select one or more first wavelengths based on said first property;
i) control said filter to filter a second multispectral reflection light signal to receive at said first camera a second signal comprising light at said one or more first wavelengths;
j) generate a first image from said filtered second multispectral light signal;
k) merge said first image with said multicolored image to create a first merged image;
l) merge said multicolored image with said monochromatic image to form a second merged image; and
m) present said first merged image and the second merged image on a display.

15. The system of claim 14, wherein filtering the one or more first wavelengths is by an optical filter, placed in front of said first camera.

16. The system of claim 14, wherein filtering the one or more first wavelengths is by selecting said one or more wavelengths from said second multispectral light signal.

17. (canceled)

18. The system of claim 14, wherein said monochromatic light source is a laser source.

19. The system of claim 14, wherein said multispectral source is a white light source.

20. The system of claim 14, further comprising:
a white light camera and wherein the controller is further configured to:
receive, at said white light camera, a white light reflection signal from said biological tissue; and
generate and present a white light image.
